(19) United States
(12) Patent Application Publication
(10) Pub. No.: US 2015/0140096 A1
(43) Pub. Date: May 21, 2015

(54) COMPOSITION AND METHOD OF PREPARATION OF BONE ALLOGRAFT FROM ENDOSTEAL PORTION OF BONE AND ISOLATED BONE PERIOSTEUM

(71) Applicant: Theodore I. Malinin, Key Biscayne, FL (US)

(72) Inventor: Theodore I. Malinin, Key Biscayne, FL (US)

(73) Assignee: VIVEX BIOMEDICAL INC., Marietta, GA (US)

(21) Appl. No.: 14/206,361

(22) Filed: Mar. 12, 2014

Related U.S. Application Data
(60) Provisional application No. 61/906,949, filed on Nov. 21, 2013.

(51) Int. Cl. A61L 27/38; A61L 27/54; A61L 27/36; A61L 27/24; A61L 27/00; A61L 27/58; A61L 27/20

(52) U.S. Cl. CPC A61L 27/3847; A61L 27/58; A61L 27/54; A61L 27/20; A61L 27/24; A61L 27/00; A61L 27/3687; A61L 27/3691; A61L 2430/40; A61L 2430/02
USPC 424/489; 424/93.7

(57) ABSTRACT

Particulate bone and structural bone, more specifically decalcified bone from endosteum having osteoinductive capacity and methods for their isolation and production are provided, more specifically, decalcified bone preparations derived from endosteal layer of bone are provided. Compositions formulated with material from endosteal layer of bone are provided along with preparation and methods of using decalcified endosteal compositions are disclosed. Endosteum is avascular and areolar in distinction to bone marrow which fills the canal. Because of its structure and anatomic position, endosteum is difficult to isolate and recognize, and therefore not regarded as an important constituent of allograft materials. One embodiment of the present invention allows for recognition and isolation of endosteum.

### TECHNICAL FIELD

The present invention relates generally to bone allograft compositions and more specifically to decalcified bone having increased osteoinductive capacity and methods for its production.

### BACKGROUND OF THE INVENTION

Mammalian bone is known to contain complex protein materials active during growth and bone healing. These induce developmental cascade of cellular events stimulating endosteal bone formation. These bone growth factors are generally referred to as bone morphogenetic (alternatively, morphogenic) proteins (BMPs). These factors have been also termed bone growth factors, bone inductive proteins, osteogenic proteins and osteoinductive proteins. The general term used in the literature as well as in this application is osteoinductive factors. Osteoinductive factors under proper circumstances initiate the differentiation of pluripotent mesenchymal cells into osteoprogenitor cells. These factors are found in very low concentration in cortical bone, but are more abundant in endosteum and periosteum. Therefore, bone grafts prepared from these tissues would stimulate healing and regeneration of bone more rapidly and effectively than would preparations from bone cortex. As demonstrated by Urist, decalcification or partial decalcification does not inhibit the activity of these factors.

Endosteum is a cellular layer which lines the medullary cavity and includes a thin layer of trabecular bone. This layer is active during bone growth, repair, fracture healing and remodeling. It covers the trabeculae of spongy bone which lay adjacent to cortical bone and line the inner surfaces of central canals. However, it is distinct from periosteum contributing as an independent and unique anatomical entity.

Endosteum is avascular and areolar in distinction to bone marrow which fills the canal. Because of its structure and anatomic position, endosteum is difficult to isolate and recognize, and therefore not regarded as an important constituent of allograft materials. One embodiment of the present invention allows for recognition and isolation of endosteum.

A bone regenerating composition or allograft or xenograft is used to heal the defects caused by trauma, pathologic conditions, surgical excisions, dental disease or other defects involving surgical corrections. Bone reconstruction may be performed with structural or particulate grafts in the form of dry or wet particles of various sizes, (jells) gels, pastes or putty containing donated human cadaveric bone or bone from animals. Since the bone is obtained and processed in advance of its use it can be preserved by freeze drying, hypothermic dehydration, chemical dehydration or by various methods of freezing and storage at temperatures from −4 degrees C. to −196 degrees C. Freeze dried bone can undergo physical processing such as grinding, milling and shaping. It can be then decalcified or demineralized. Complete or extreme demineralization results in the preparation of demineralized bone matrix (DBM). Preparations and alleged virtue of DBM had been disclosed in several US Patents (U.S. Pat. No. 4,394,370, U.S. Pat. No. 4,440,750, U.S. Pat. No. 4,485,097, U.S. Pat. No. 4,678,470, U.S. Pat. No. 4,743,259 and U.S. Pat. No. 8,435,566). Regardless of degree of demineralization or lack thereof, the primary objective in bone regeneration is that the transplanted material be effective in inducing bone formation, become incorporated into the application site and result in new bone formation. The biological properties of bone transplants are determined by several factors including the anatomical site from which the transplants are taken, physical and chemical characteristics of the donor tissue and the methods used to prepare, conserve and store tissue prior to transplantation. Much work had been done on bone induction and the method to maximize osteoinductive properties of allografts. In this regard decalcification and seminal work of Marshall Urist is cited. (Urist 1983 a and b). However, Urist never promotes completely demineralized bone (DBM), but only partially demineralized or surface demineralized bone. It is now known that complete demineralization of bone leads to the loss of its osteoinductive properties (Pietrzak et al 2011).

### SUMMARY OF THE INVENTION

This invention relates to bone regenerating composition and methods of its production and use.

Endosteal and periosteal composition specifically particulate and membranous having osteoinductive capacity and methods of their preparation are provided. Endosteum is a layer of mesenchymal connective tissue that lines the medullary canals of bone (internal surface). Connective tissue cells of endosteum line the trabeculae of spongy bone adjacent to the cortical bone. Endosteal compositions may contain either a combination of trabecular bone lined with endosteal cells or segregated endosteal cells. The periosteal layer of cells is a membrane which can be prepared and preserved as such or it can be particularized. The cells can be also segregated and preserved as such. The method of preparation of the periosteum is distinctly unique and different from those known in the art.

In one embodiment, a bone repair composition has particulate composition or membranous composition of cells, the particulate composition having a higher percentage of particles from an endosteum cellular layer or endosteal bone.

In another embodiment, a bone repair isolated periosteal tissue composition has particulate composition or membranous composition, the particulate composition having a higher percentage of particles from a periosteum cellular layer or periosteal bone. Preferably, the periosteal particles or membranes are prepared from the periosteum alone, not including subperiosteal cortical bone. The particles or the composition can be preserved by freeze-drying.

In another embodiment, the bone repair composition of particulate or membranous whether using endosteum cellular layer or endosteal bone or isolated periosteal tissue composition of particulate membrane or combination thereof can be preserved by hypothermic dehydration.

The compositions of the various embodiments can be demineralized bone, or partially decalcified wherein said composition is surface decalcified. Said compositions can be decalcified by citric acid, ethylenediamine, tetraacetic acid, hydrochloric acid, electrolytic process or a combination thereof. Bone repair composition can have said particles measure 1 to 1000 microns. In a preferred embodiment, endosteal cells are dispersed and separated from the trabecular bone. The endosteal cells can be preserved by cryopreservation, freezing, freeze-drying, hypothermic dehydration or chemical preservation. The segregated endosteal cells can be intermixed with particulate bone or are seeded on structural bone grafts to form the bone repair composition.

In another embodiment, the bone particles or cells can be suspended in a solution of hydroxyethyl starch in a form of jelly, liquid, putty or paste. Alternatively, the bone particles or cells can be suspended in human or animal collagen solution comprising a liquid, jelly, putty or paste. Optionally, the bone particles or cells can be suspended in a solution of sucrose comprising a liquid, jelly, paste or putty. In another embodiment, the particles or cells are mixed with a group of liquids consisting of physiological salt solution, lactated Ringer's solution, any of the accepted balanced salt solutions and or blood products.

Bone repair composition can be made by separating endosteum from long bone cortex occurs without resorting to milling which includes cortical bone. The endosteum when separated from long bone is processed under aseptic conditions and processed aseptically. The endosteum is identified and separated from freeze-dried bone by the action of citric acid, ethylenediamine tetraacetic acid, hydrochloric acid, electrolytic demineralization or a combination thereof.

In the embodiments having a composition made of periosteal particles and membranous, periosteal cells are dispersed and separated from the trabecular bone and periosteal cells are preserved by cryopreservation, freezing, freeze-drying, hypothermic dehydration or chemical preservation. The segregated periosteal cells can be intermixed with particulate bone or are seeded on structural bone grafts.

In the embodiments, separating the periosteum from long bone cortex occurs without resorting to milling which includes cortical bone, under aseptic conditions and processed aseptically. The periosteum is identified and separated from freeze-dried bone by the action of citric acid, ethylenediamine tetraacetic acid, hydrochloric acid, electrolytic demineralization or a combination thereof.

In still another embodiment, periosteum is prepared as a fluff composition of branching strands 400 to 2000 microns in length.

### BRIEF DESCRIPTION OF THE DRAWINGS

The invention will be described by way of example and with reference to the accompanying drawings in which:

FIG. 1 is a photograph of treated long bone interior with arrows pointing to the endosteum.

FIG. 2 is a photograph of the endosteum separated and preserved as an intact structure.

FIG. 3A is a photograph of an exemplary fluff composition made according the present invention.

FIG. 3B is a photograph of a magnified exemplary fluff composition made according the present invention.

### DETAILED DESCRIPTION OF THE INVENTION

The following description of technology is exemplary in nature of the subject matter, manufacture and utilization of one or more inventions, and is not intended to limit the scope or uses of any inventions obtained in this application. The present invention provides bone repair compositions for applications to the surface of bone or internally into bone defects of a human or animal subject. The bone repair composition has particulate matter or membranous structure, or preferably both. The particulate matter or membranous structure has a higher percentage concentration of endosteum or endosteal bone than other bone constituents in one embodiment. In an alternative embodiment, a higher percentage of periosteum is employed.

The compositions of disclosed technology may be provided in any form suitable for transplantation into bone of the recipients. Bone allografts gained in popularity when they became available through tissue banks There are varieties of grafting materials available. Freeze-dried and frozen allografts are the most common. When bone allografts which retain bone morphogenic proteins (BMPs) are placed in contact with vascularized host bone, healing occurs when the calcified matrix is replaced by new bone. The individual peculiarities of the human skeleton are such that each bone has its own requirements for healing, immobilization and bone grafting. Therefore, there is no universal all-purpose bone allograft and there is no single way for preparing all bone allografts. To date, the most successful bone allografts have been aseptically excised and aseptically processed freeze-dried cortical cancellous and corticocancellous grafts. However, success with bone transplantation had been qualified. Therefore search for new and improved methods for allograft preparations and utilization continues. The present invention is part of this search.

Bone allografts commonly used in reconstructive procedures of the skeletal system can be broadly divided into particulate and structural grafts. The former are used most frequently. Particulate grafts can be crushed cancellous or cortical bone (bone chips), ground bone, morselized bone or microparticulate grafts. These are used for filling bone defects with largely intact walls. The structural grafts are bone plates (bone struts), sections of mandibles, bone blocks, bone shaft segments, wedges, ribs, etc. Bone blocks of various sizes and shapes are used to support implants and to obliterate bone voids.

Bone allografts must possess osteoinductive properties that are maintained by some methods of preservation, reduced by some and destroyed by others. Morphologic analysis of allogenic bone grafts removed from patients before complete healing and incorporation shows them to be largely acellular. The graft is surrounded by host mesenchymal tissue that undergoes metaplasia and ossification. This basically outlines the entire spectrum of bone allograft interaction with the host. Aside from temporal considerations no quantitative differences have been noted between the allograft and freeze-dried allografts. To a lesser degree, the same patterns have been noted in frozen allografts incorporation as healing progresses through revascularization, osteoclastic resorption, new bone formation and remodeling. The response to allograft implantation is modified by the ways in which the grafts are prepared. There are a number of methods that may include irradiation, exposure to chemicals, etc., but aseptically excised and processed allografts serve as a standard.

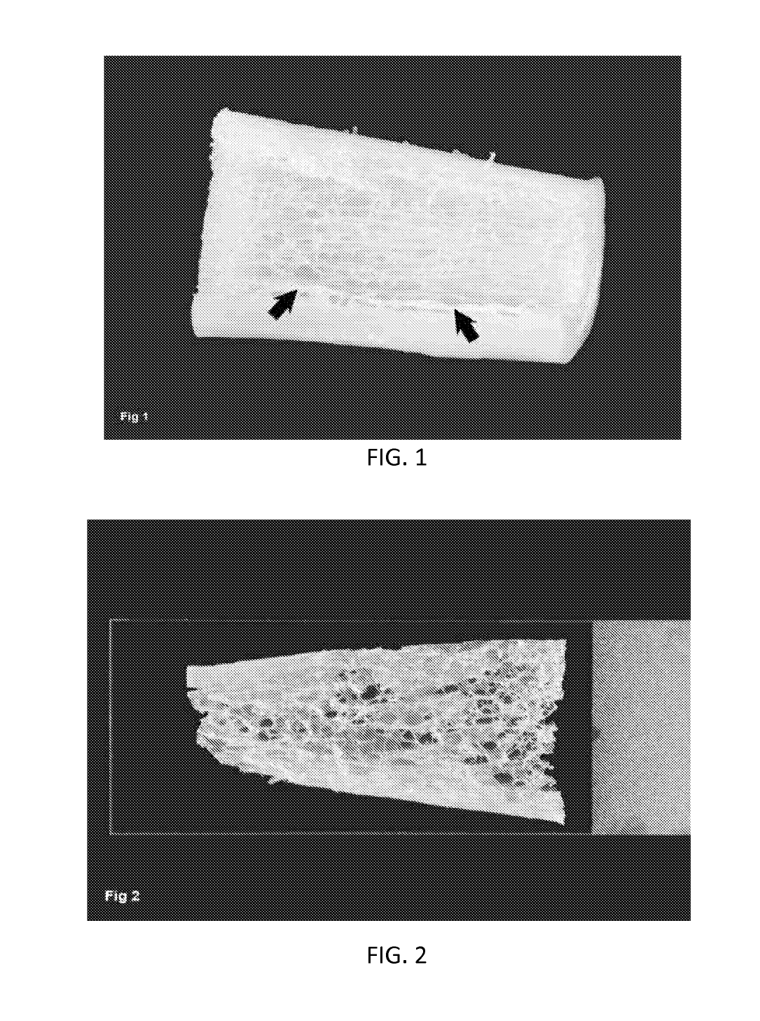

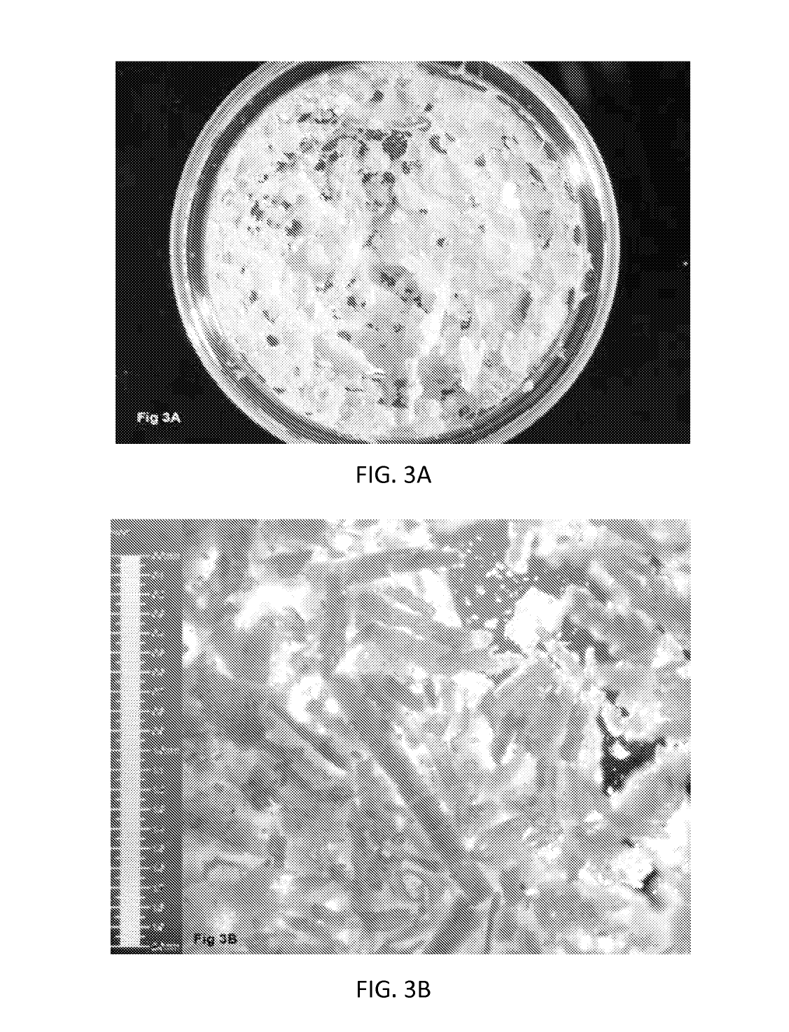

Although freeze-drying had been used with considerable success in bone allograft preservation for over 75 years, the process has some distinct disadvantages as well as advantages. The disadvantages include severe morphological alterations in freeze-dried tissues. These have been attributed not as much to freeze-drying itself, but to freezing which must precede freeze-drying and is maintained through most of the process to avoid freezing damage as well as the associated phenomena, the present embodiment discloses desiccation at hypothermia. The inventor had used this process for the last several years in the perseveration of cartilage with results superior to those obtained with freeze-drying. The process is relatively simple. While with freeze-drying, tissue is first frozen solid and then dehydrated in vacuum, hypothermic dehydration depends on placing the object at reduced temperatures above freezing point into a high vacuum chamber allowing it to dry to a desired residual moisture level. The result is dried tissue without fissures, microscopic ice crystal distortion and collapse phenomenon. Biological advantage of freeze-drying is that it alters protein structures to allow collagen extraction by heating or other biochemical processes. The present embodiment utilized this property by placing segments of freeze-dried bone from whose medullary canals bone marrow, both red and yellow, had been removed by steel brushes and lavage with saline into solutions of citric acid, EDTA and hydrochloric acid. These treatments rendered endosteum clearly visible as a distinct structure, as shown in FIG. 1, allowing for its removal with curved chisels, gauges, or similar instrument. This yielded the product of pure endosteum without accompanying cortical bone inherent in preparations obtained by milling or sawing or similar processes. Once endosteum is separated, it is either re-freeze-dried or hypothermically dehydrated and then micronized or preserved as an intact structure, as shown in FIG. 2. The same principle applies to periosteum. In still another embodiment, periosteum is prepared as a fluff composition of branching strands 400 to 2000 microns in length, as illustrated in FIGS. 3A and 3B. Alternatively, the endosteum could be prepared as a fluff composition.

Material in the compositions disclosed above may be obtained from any animal sources. Material obtained from animals may be used for xenogenic transplantation into humans. However, preferably for human use bone is obtained from deceased or living human donors. Tissues from these donors are available from a number of tissue banks, regulated by the US Food and Drug Administration. Procedures for processing and storage of human tissues are well known. Such procedures and controls thereof are appreciated by one of ordinary skill in the art.

Preferred embodiments of this invention are described herein, including the best mode known to the inventor for carrying out the invention. Of course, variations of those preferred embodiments will become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modification equivalents of the subject matter recited in the claims appended hereto as permitted by applicable law. Moreover, any combination of the-above-described elements in all possible variations thereof is encompassed by the invention unless otherwise indicated herein or otherwise clearly contradicted by context.

What is claimed is:

1. A bone repair composition comprising particulate composition or membranous composition of cells, the particulate composition having a higher percentage of particles from an endosteum cellular layer or endosteal bone.

2. Bone repair composition of claim 1 wherein particulate composition is preserved by freeze-drying.

3. Bone repair composition of claim 1 wherein said composition is preserved by freeze-drying.

4. Bone repair composition of claim 1 wherein said composition is preserved by hypothermic dehydration.

5. Bone repair composition of claim 1 wherein said composition comprises demineralized bone.

6. Bone repair composition of claim 1 wherein said composition is partially decalcified.

7. Bone repair composition of claim 1 wherein said composition is surface decalcified.

8. Bone repair composition of claim 1 wherein said composition is decalcified by citric acid, ethylenediamine, tetraacetic acid, hydrochloric acid, electrolytic process or a combination thereof.

9. Bone repair composition of claim 1 wherein said particles measure 1 to 1000 microns.

10. Bone repair composition of claim 1 wherein endosteal cells are dispersed and separated from the trabecular bone.

11. Bone repair composition of claim 1 wherein endosteal cells are preserved by cryopreservation, freezing, freeze-drying, hypothermic dehydration or chemical preservation.

12. Bone repair composition of claim 1 wherein segregated endosteal cells are intermixed with particulate bone or are seeded on structural bone grafts.

13. Bone repair composition of claim 1 wherein bone particles or cells are suspended in a solution of hydroxyethyl starch in a form of jelly, liquid, putty or paste.

14. Bone repair composition of claim 1 wherein bone particles or cells are suspended in human or animal collagen solution comprising a liquid, jelly, putty or paste.

15. Bone repair composition of claim 1 wherein bone particles or cells are suspended in a solution of sucrose comprising a liquid, jelly, paste or putty.

16. Bone repair composition of claim 1 wherein the particles or cells are mixed with a group of liquids consisting of physiological salt solution, lactated Ringer's solution, any of the accepted balanced salt solutions and or blood products.

17. Bone repair composition of claim 1 wherein separating endosteum from long bone cortex occurs without resorting to milling which includes cortical bone.

18. Bone repair composition of claim 1 wherein endosteum is separated from long bone under aseptic conditions and processed aseptically.

19. Bone repair composition of claim 1 wherein endosteum is identified and separated from freeze-dried bone by the action of citric acid, ethylenediamine tetraacetic acid, hydrochloric acid, electrolytic demineralization or a combination thereof.

20. A bone repair isolated periosteal tissue composition comprising particulate composition or membranous composition, the particulate composition having a higher percentage of particles from a periosteum cellular layer or periosteal bone.

21. Bone repair composition of claim 20 wherein periosteal particles or membranes are prepared from the periosteum alone, not including subperiosteal cortical bone.

22. Bone repair composition of claim 21 wherein particulate composition is preserved by freeze-drying.

23. Bone repair composition of claim 21 wherein said composition is preserved by freeze-drying.

24. Bone repair composition of claim 23 wherein said composition is preserved by hypothermic dehydration.

25. Bone repair composition of claim 20 wherein the composition is prepared from periosteum subjected to hypothermic dehydration.

26. Bone repair composition of claim 20 wherein said composition comprises demineralized bone.

27. Bone repair composition of claim 20 wherein said composition is partially decalcified.

28. Bone repair composition of claim 20 wherein said composition is surface decalcified.

29. Bone repair composition of claim 20 wherein said composition is decalcified by citric acid, ethylenediamine, tetraacetic acid, hydrochloric acid, electrolytic process or a combination thereof.

30. Bone repair composition of claim 20 wherein said particles measure 1 to 1000 microns.

31. Bone repair composition of claim 20 wherein periosteal cells are dispersed and separated from the trabecular bone.

32. Bone repair composition of claim 20 wherein periosteal cells are preserved by cryopreservation, freezing, freeze-drying, hypothermic dehydration or chemical preservation.

33. Bone repair composition of claim 20 wherein segregated periosteal cells are intermixed with particulate bone or are seeded on structural bone grafts.

34. Bone repair composition of claim 20 wherein bone particles or cells are suspended in a solution of hydroxyethyl starch in a form of jelly, liquid, putty or paste.

35. Bone repair composition of claim 20 wherein bone particles or cells are suspended in human or animal collagen solution comprising a liquid, jelly, putty or paste.

36. Bone repair composition of claim 20 wherein bone particles or cells are suspended in a solution of sucrose comprising a liquid, jelly, paste or putty.

37. Bone repair composition of claim 20 wherein the particles or cells are mixed with a group of liquids consisting of physiological salt solution, lactated Ringer's solution, any of the accepted balanced salt solutions and or blood products.

38. Bone repair composition of claim 20 wherein separating periosteum from long bone cortex occurs without resorting to milling which includes cortical bone.

39. Bone repair composition of claim 20 wherein periosteum is separated from long bone under aseptic conditions and processed aseptically.

40. Bone repair composition of claim 20 wherein periosteum is identified and separated from freeze-dried bone by the action of citric acid, ethylenediamine tetraacetic acid, hydrochloric acid, electrolytic demineralization or a combination thereof.

41. Bone repair composition of claim 20 wherein periosteum is prepared as a fluff composition of branching strands 400 to 2000 microns in length.
